(19) United States
(12) Patent Application Publication
(10) Pub. No.: US 2021/0137382 A1
(43) Pub. Date: May 13, 2021

(54) Personnel Monitoring and Reporting System

(71) Applicant: Northern Applied Technologies, LLC, Grand Forks, ND (US)

(72) Inventor: Brandon J. Koster, Grand Forks, ND (US)

(73) Assignee: Northern Applied Technologies, LLC

(21) Appl. No.: 16/678,095

(22) Filed: Nov. 8, 2019

(51) Int. Cl. A61B 5/00; A61B 5/024; G08B 25/01; G08B 21/04; A41D 1/00; H04W 4/02; H04W 4/80; F41A 33/02

(52) U.S. Cl. CPC A61B 5/0024; A61B 5/6805; A61B 5/02438; A61B 5/681; F41A 33/02; G08B 21/0453; A41D 1/005; H04W 4/026; H04W 4/80; G08B 25/016

(57) ABSTRACT

A personnel monitoring and reporting system for monitoring, reporting, and displaying real-time field data including personnel health status, location, role, and other information. The personnel monitoring and reporting system generally includes a personnel system including a computing unit which is worn by each operator in the field. The computing unit may include sensors and a rip cord for indicating health status of the operator. The computing unit may be connected to a personnel display unit worn on the wrist of the operator with a display screen which displays health status, location, and other information relating to both the operator and team members. The computing unit may also be connected to a laser targeting system equipped to a weapon for highlighting targets of interest.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND

### Field

Example embodiments in general relate to a personnel monitoring and reporting system for monitoring, reporting, and displaying real-time field data including personnel health status, location, role, and other information.

### Related Art

Any discussion of the related art throughout the specification should in no way be considered as an admission that such related art is widely known or forms part of common general knowledge in the field.

During law enforcement, military, and other security operations, teams of personnel are often deployed in various locations to perform various duties. These teams of personnel may often operate in different areas of certain locations. For example, teams of personnel may be deployed to a complex of buildings, with different team members being assigned to clear different buildings. As another example, teams of personnel may be deployed to a large, forested area where team members are not within sight or sound of each other.

In such situations, it would be preferable to have a unified system to monitor, report, and display real-time field data relating to the personnel in the field. While radio and GPS systems have been used in the past to monitor and report personnel information, such systems often suffer from a number of shortcomings. Centralized network communication portals such as cellular towers may be compromised or non-existent in the area of interest which can compromise communications. Further, in high-intensity situations, it can detrimental to rely on self-reporting of units in the field for health conditions such as high stress, injury, or even death. Additional, in areas of low-light or limited visibility, it can be difficult for team members to keep track of the locations of other personnel—increasing the risk of blue-on-blue incidents.

### SUMMARY

An example embodiment is directed to a personnel monitoring and reporting system. The personnel monitoring and reporting system includes a personnel system including a computing unit which is worn by each operator in the field. The computing unit may include sensors and a rip cord for indicating health status of the operator. The computing unit may be connected to a personnel display unit worn on the wrist of the operator with a display screen which displays health status, location, and other information relating to both the operator and team members. The computing unit may also be connected to a laser targeting system equipped to a weapon for highlighting targets of interest.

There has thus been outlined, rather broadly, some of the embodiments of the personnel monitoring and reporting system in order that the detailed description thereof may be better understood, and in order that the present contribution to the art may be better appreciated. There are additional embodiments of the personnel monitoring and reporting system that will be described hereinafter and that will form the subject matter of the claims appended hereto. In this respect, before explaining at least one embodiment of the personnel monitoring and reporting system in detail, it is to be understood that the personnel monitoring and reporting system is not limited in its application to the details of construction or to the arrangements of the components set forth in the following description or illustrated in the drawings. The personnel monitoring and reporting system is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a front view of a personnel system of a personnel monitoring and reporting system in accordance with an example embodiment.

FIG. 2 is a front view of a personnel system with rip cord removed of a personnel monitoring and reporting system in accordance with an example embodiment.

FIG. 3 is a front view of a personnel system secured to body armor of a personnel monitoring and reporting system in accordance with an example embodiment.

FIG. 4 is a perspective view of a personnel system secured to body armor of a personnel monitoring and reporting system in accordance with an example embodiment.

FIG. 5 is a block diagram of an exemplary computing unit of a personnel monitoring and reporting system in accordance with an example embodiment.

FIG. 6 is a bottom view of a computing unit of a personnel monitoring and reporting system in accordance with an example embodiment.

FIG. 7 is a bottom perspective view of a computing unit of a personnel monitoring and reporting system in accordance with an example embodiment.

FIG. 8 is a block diagram illustrating interconnection between the computing unit and the rip cord of a personnel monitoring and reporting system in accordance with an example embodiment.

FIG. 9 is a top perspective view of a computing unit of a personnel monitoring and reporting system in accordance with an example embodiment.

FIG. 10 is a perspective view of a personnel monitoring and reporting system in use in accordance with an example embodiment.

FIG. 11 is a perspective view of a personnel display unit worn on the wrist of an operator of a personnel monitoring and reporting system in accordance with an example embodiment.

FIG. 12 is a front view of a personnel display unit of an operator of a personnel monitoring and reporting system in accordance with an example embodiment.

FIG. 13 is a side view of a personnel display unit illustrating ports and connectors of a personnel monitoring and reporting system in accordance with an example embodiment.

FIG. 14 is a frontal view and block diagram illustrating the connection between a processor of a computing unit and the personnel display unit of a personnel monitoring and reporting system in accordance with an example embodiment.

FIG. 15 is a block diagram illustrating interconnection between a processor of a computing unit and a personnel display unit of a personnel monitoring and reporting system in accordance with an example embodiment.

FIG. 16 is a front view of an exemplary display screen of a personnel display unit of a personnel monitoring and reporting system in accordance with an example embodiment.

FIG. 17 is a side view of a firearm equipped with a laser targeting system of a personnel monitoring and reporting system in accordance with an example embodiment.

FIG. 18 is a side view of a laser targeting system of a personnel monitoring and reporting system in accordance with an example embodiment.

FIG. 19 is a block diagram of a laser targeting system of a personnel monitoring and reporting system in accordance with an example embodiment.

FIG. 20 is a diagram illustrating a mesh network of a personnel monitoring and reporting system in accordance with an example embodiment.

FIG. 21 is a block diagram illustrating a communications network of a personnel monitoring and reporting system in accordance with an example embodiment.

FIG. 22 is a block diagram illustrating a mesh network of a personnel monitoring and reporting system in accordance with an example embodiment.

FIG. 23 is a flowchart illustrating mesh and mirror relations of a personnel monitoring and reporting system in accordance with an example embodiment.

FIG. 24 is a block diagram of a cellular network of a personnel monitoring and reporting system in accordance with an example embodiment.

FIG. 25 is a front view of an exemplary personnel display unit of a personnel monitoring and reporting system in accordance with an example embodiment.

### DETAILED DESCRIPTION

### A. Overview

An example personnel monitoring and reporting system 10 generally comprises a computing unit 21 adapted to be secured to an operator 12, the computing unit 21 comprising a processor 100, 101, 109 and a transceiver 27, wherein the computing unit comprises a pair of electrical contacts 34a, 34b. A physiological sensor 67 is communicatively interconnected with the computing unit 21, wherein the physiological sensor 67 is adapted to be in physical contact with the operator 12, wherein the physiological sensor 67 is adapted to transmit one or more health conditions of the operator to the computing unit 21.

A rip cord 40 is removably connected to the computing unit 21, wherein the rip cord 40 comprises a conductive strip 48 adapted to electrically connect the pair of electrical contacts 34a, 34b of the computing unit 21, wherein the rip cord 40 is adapted to be removed from the computing unit 21 such that the conductive strip 48 no longer electrically connects the pair of electrical contacts 34a, 34b of the computing unit 21, and wherein the computing unit 21 is adapted to transmit a distress signal through the transceiver 27 when the pair of electrical contacts 34a, 34b are not electrically connected by the conductive strip 48.

The computing unit 21 may comprise a sensor port 113, wherein the physiological sensor 67 is connected to the sensor port 113 of the computing unit 21 by a sensor cable 66. The physiological sensor 67 may comprise a pulse detector such as an EKG pad. The computing unit 21 may comprise a memory slot 112 for receiving a memory card 102, the memory card 102 being adapted to update software of the computing unit 21. The transceiver 27 may be comprised of a Bluetooth transceiver, a radio antenna, or a cellular communications unit. The computing unit 21 may comprise a first power port 111 and the personnel display unit 50 may comprise a second power port 56 interconnected by a power cable 64 such that the personnel display unit 50 is powered by the computing unit 21. The computing unit 21 may further comprise a first signal port 110 and the personnel display unit 50 may further comprise a second signal port 55 interconnected by a signal cable 62 such that the computing unit 21 controls a display screen 52 of the personnel display unit 50.

A personnel display unit 50 may be communicatively interconnected with the computing unit 21, wherein the personnel display unit 50 is adapted to be worn by the operator 12. The personnel display unit 50 may comprise a display screen 52 for displaying information about the operator 12. The display screen 52 may be further adapted to display a status and location of one or more team member operators 12 each operating a secondary computing unit 21. Generally, the display screen 52 will not display status and location of anyone in the field not operating a computing unit 21. The personnel display unit 50 may be worn on the wrist of the operator 12.

The personnel monitoring and reporting system 10 may also comprise a laser targeting system 70 connected to the weapon 16, the laser targeting system 70 comprising a laser emitter 72 such that the operator 12 may identify one or more targets with the laser targeting system 70. The laser targeting system 70 may comprise a laser range finder 73 comprised of a laser receiver for detecting a range to any of the one or more targets identified with the laser targeting system 70. The laser targeting system 70 may include an electronic compass and may be wirelessly connected to the computing unit 21, such as via Bluetooth.

A method of monitoring personnel during an operation may comprise the steps of equipping a plurality of operators 12 with a plurality of computing units 21, wherein each of the operators 12 wears one of the plurality of computing units 21, wherein each of the plurality of computing units 21 comprises a transceiver 27 having an effective range; identifying any of the plurality of computing units 21 in the effective range by a first computing unit 21 of the plurality of computing units 21; transmitting a location information of any of the plurality of computing units 21 identified in the effective range by the transceiver 27 of the first computing unit 21 to a squad reporting radio 130; and transmitting the location information of any of the plurality of computing units 21 identified in the effective range by the squad reporting radio 130 to a central control unit such as a network computer 134.

The first computing unit 21 may transmit the location information of any of the plurality of computing units 21 not in the effective range of the first computing unit 21 to the squad reporting radio 130 by the central control unit 134. The squad reporting radio 130 may also transmit the location information of any of the plurality of computing units 21 not in the effective range of the first computing unit 21 to the first computing unit 21.

### B. Personnel Asset Computer

As shown in FIGS. 1-5, a personnel system 20 may be secured to the body of an operator 12. The personnel system 20 is utilized to collect real-time information about the operator including health status. Health status and other information may be detected, such as by one or more physiological sensors 67. In addition or alternatively, certain operator information may be manually entered. Other functions include reporting location (such as through GPS), processing information from other components of the system 10, such as but not limited to a personnel display unit 50 and/or laser targeting unit 70.

The personnel system 20 may include a computing unit 21, sometimes referred to as “personnel asset computer” or “PAC”, which is worn on the operator's 12 clothing, such as on a belt, vest, or body armor 13. The computing unit 21 may include an outer covering 30 such as a pouch in which the computing unit 21 is partially or fully positioned so as to protect the computing unit 21 from damage and the elements.

The computing unit 21 may include one or more processors 100, 101, 109, such as an ARM processor 109 or serially-connected dual processors 101, 101 for the processing of all functions of the computing unit 21. The computing unit 21 may run software from secondary memory while processing information from one or more physiological sensors 67 and reporting through a series of command lines through a network. Cell phone and radio antennas 27 may also be included for the reporting of information and data through a cellular phone network or by line of sight radio signal, depending on the needs of the operators 12. In some embodiments, multiple communications protocols may be utilized. For example, cell phone communications may be reserved for updating the application server while RF24 radio communications are reserved for team communications. The computing unit 21 may also include a GPS module 107 which performs a serial uplink to the processor 101, 101, 109 to communicate the operator's 12 current longitude and latitude. This may be utilized for pin-pointing the physical locations of any operators 12 using the personnel system 20. The location information may be continuously sent through the network as it becomes available from the GPS module 107.

To determine health status, a sensor 67 circuit and rip cord 40 may also be installed with the personnel system 20. The sensor 67 may constantly monitor the operator's 12 vital health information, such as but not limited to pulse, to determine if the operator 12 is alive and detect any potential injuries or health conditions that may need attention. The rip cord 40 may be pulled by the operator 12 as discussed below if the operator 12 is injured or needs assistance. Both the sensor 67 and rip cord 40 are utilized to determine the health status of the operator 12. If, for example, the sensor 67 does not detect a pulse, the personnel system 20 may report that the operator has been possibly killed in action.

FIGS. 3 and 4 illustrate an exemplary sensor 67 comprised of three EKG pads 67a, 67b, 67c. Each of the EKG pads 67a, 67b, 67c may be removably secured to the body of the operator 12 to detect pulse and/or other health conditions of the operator 12 when in the field. In the exemplary embodiment shown in FIGS. 3 and 4, a first EKG pad 67a, a second EKG pad 67b, and a third EKG pad 67c is shown. Generally, the second EKG pad 67b will be labelled as “con” such that the second EKG pad 67b is always in the middle, with the first and third EKG pads 67a, 67c being positioned on either side of the second EKG pad 67b. It should be appreciated that other types of sensors 67 or EKG pads 67a, 67b, 67c may be utilized in different embodiments to sense different physiological conditions of the operator 12.

The computing unit 21 may also support Bluetooth communications through a Bluetooth module 104. Bluetooth communications may be utilized to integrate the computing unit 21 with other components of the system 10 wirelessly to prevent or limit reliance on bulky wires and cables. The Bluetooth module 104 may be embedded in the main circuit of the computing unit 21.

For integration of the personnel display unit 50, the computing unit 21 may include both a signal port 110 and a power port 111 as discussed below. Secondary memory in the computing unit 21 may also integrate with the graphics card in the personnel display unit 50 to allow for rending of graphics to the display screen 52 of the personnel display unit 50.

The personnel display unit 50 is not directly mounted to the computing unit 21, but instead may be worn on the wrist of the operator 12 while receiving processing and software functions via the signal port 110 of the computing unit 21. The personnel display unit 50 is thus integrated through a signal cable 62 to allow for the computing unit 21 to provide the complex signal and software functions necessary for use. A software package performs all of the functions of the computing unit 21 including the calculation of data from server packages, launching field networks, rendering graphics for the personnel display unit 50, and reporting information back to the application server.

The computing unit 21 may be worn on the vest or body armor 13 of the operator 12 and wirelessly transmits personnel information through one or more networks, such as cellular networks or RF24 short-range networking. This information may include the operator's 12 individual identification information, health status, location, and targeting information.

The computing unit 21 includes its own processor(s) 100, 101, 109 attached to a GPS module 107, sensor 67 circuits, secondary memory, and radio transmitters and receivers for both cell phone and radio use. On-board software may determine the health status of the operator 12, generate images for the personnel display unit 50, and integrate other sub-components of the personnel system 20 such as the laser targeting system 70 described herein. The computing unit 21 may rely on sensor packages such as heart rate sensors, GPS modules 107, and secondary memory such as SD cards 102. With this information, the software in the computing unit 21 may calculate the information and ready data for network use.

The computing unit 21 primarily determines health status and location throughout the network. The computing unit 21 may rely on different communications protocols depending on the type of use. For example, for law enforcement usage, cell phone towers may be used to transmit information to the application server. For military usage, long range transmitters and military standard communication security may be utilized. Hybrid communications protocols may also be utilized as discussed herein. In some embodiments, the computing unit 21 may accommodate multiple communications protocols, with the operator 12 having the option to select one or more communications protocols for a specific operation.

FIGS. 1, 2, and 5-9 best illustrate an exemplary personnel unit 20 for use with the system 10. As best shown in FIGS. 7 and 9, the personnel unit 20 may comprise a housing including an upper end 22, a lower end 23, a front end 24, and a rear end 25. The personnel unit 20 will generally comprise the computing unit 21 and outer covering 30, both of which may be secured to the body of the operator 12, such as to body armor 13 as shown in the exemplary figures.

The computing unit 21 will generally comprise a rectangular-shaped housing such as shown in the figures, though other configurations may be utilized. The outer covering 30 may comprise a sleeve, pocket, wrap, or other fabric-type covering that fully or partially covers the computing unit 21 so as to prevent the computing unit 21 from blunt-impact damage or the elements.

As shown in FIG. 1, the front end 24 of the personnel system 20 may comprise fasteners 32 such as hook-and-loop fasteners for removably securing various items, such as but not limited to patches 14 and a rip cord 40, to the personnel system 20. FIG. 3 illustrates a patch 14 which has been affixed to the front end 24 of the personnel system 20 over the fasteners 32. Such patches 14 may be utilized to identify the nationality of the operator 12, or for use of personal effects of the operator 12.

As best shown in FIGS. 1 and 2, a rip cord 40 may be removably connected to the personnel system 20 so that the operator 12 may quickly indicate the need for assistance by pulling the rip cord 40. As shown in FIG. 2, the rip cord 40 may be removably connected to the fasteners 32 on the front end 24 of the personnel system 20 such that the rip cord 40 may be easily pulled away and disconnected from the personnel system 20 when needed.

The rip cord 40 may comprise a rectangular-shaped member which is removably affixed to the fasteners 32 on the front end 24 of the personnel system 20. It should be appreciated, however, that the shape, size, and configuration of the rip cord 40 may vary in different embodiments. As shown in the figures, the rip cord 40 may comprise an upper end 41, a lower end 42, a front end 43, and a rear end 44.

The rip cord 40 may be removably connected to the computing unit 21 and/or outer covering 30 of the personnel system 20. As shown in FIG. 2, the rear end 44 of the rip cord 40 may include a rip cord fastener 46 comprised of hook-and-loop fasteners which are adapted to removably engage with the corresponding fasteners 32 on the front end 24 of the computing unit 21.

The front end 24 of the personnel system 20 may include one or more electrical contacts 34a, 34b comprised of a conductive material such as shown in FIG. 2. It should be appreciated that the shape, number, and configuration of the electrical contacts 34a, 34b may vary in different embodiments and thus should not be construed as limited by the exemplary embodiment shown in FIG. 2. In the exemplary embodiment shown in FIG. 2, a first contact 34a is positioned at a horizontally-displaced position with respect to a second contact 34b on the front end 24 of the personnel system 20.

As shown in FIG. 2, when the rip cord 40 is affixed to the personnel system 20, the contacts 34a, 34b will be linked together by the conductive strip 48 on the rear end 44 of the rip cord 40 to close an electrical circuit. When the rip cord 40 is removed, the conductive strip 48 will no longer electrically link the first contact 34a with the second contact 34b, thus opening the circuit to indicate an emergency situation with respect to the operator 12.

There are numerous examples of situations in which the physiological sensor 67 may not detect any abnormality while the operator 12 is in a dangerous situation. For example, an operator 12 who is pinned down in a location surrounded by enemy units may not have any indicators present in his/her physiological condition(s) detected by the physiological sensor(s) 67. In such a case, the operator 12 may pull the rip cord 40, thus removing the conductive strip 48 from electrically connecting the two contacts 34a, 34b to indicate that the operator 12 needs assistance.

The computing unit 21, upon detecting that the rip cord circuit has been opened by removal of the rip cord 40, will transmit a message to the central server or other personnel to assist the operator 12 who pulled the rip cord 40. This information may include identifying information of the operator 12 in danger, the physiological condition of the operator 12, and/or the location of the operator 12 in the field.

FIG. 7 illustrates an exemplary manner for interconnecting the computing unit 21 with a rip cord 40 such that the computing unit 21 will detect when the rip cord 40 has been removed. In the exemplary embodiment shown in FIG. 7, it can be seen that the computing unit 21 has been fitted with a J2 port 36. The J2 port 36 is linked with one or more processors 100, 101, 109 of the computing unit 21. A cable (not shown) may be utilized to interconnect the J2 port 36 of the computing unit 21 with the rip cord 40.

The configuration shown in FIGS. 7 and 9 is merely an exemplary embodiment shown for illustrative purposes. It should be appreciated that the positioning, orientation, size, and configuration of any ports on the computing unit 21 for interfacing with the rip cord 40 may vary in different embodiments. In some embodiments, the rip cord 40 may be connected by contact rather than conduit. Further, the type of port used may vary and should not be construed as limited to the J2 port 36 design shown in the exemplary embodiment of FIGS. 7 and 9.

FIG. 8 illustrates an exemplary interconnection between the computing unit 21 and the rip cord 40. As can be seen, a cable is used to connect between the J2 port 36 of the computing unit 21 and the rip cord 40. The use of a J2 port 36 on the computing unit 21 allows for a wide range of different rip cord 40 designs to be utilized with the personnel monitoring and reporting system 10. The rip cord 40 will generally have its own port (not shown) that may electrically link the rip cord 40 with the computing unit 21 such that the computing unit 21 may detect when the electrical contacts 34a, 34b are not connected by the conductive strip 48.

As shown in FIG. 1, the rip cord 40 may also include a tab 45 extending from its lower end 42. The tab 45 may comprise a projection extending downwardly from the lower end 42 of the rip cord 40 such as shown in the figures. However, it should be appreciate that the size, shape, configuration, and placement of the tab 45 may vary in different embodiments. Generally, the tab 45 will provide an easy grasping point for the operator 12 to pull the rip cord 40 so as to open the rip cord circuit and transmit a request for assistance.

An exemplary embodiment of the computing unit 21, also referred to as a personnel asset computer (PAC), can be seen in FIGS. 1-9 of the drawings. The computing unit 21 will generally comprise a rectangular-shaped housing, but other configurations may be utilized. As shown in FIG. 8, the upper end 22 of the computing unit 21 may comprise an antenna jack 26 for removably receiving an antenna 27 or transceiver for communications purposes. In some embodiments, the antenna 27 may be fixedly connected to the computing unit 21. In other embodiments, the antenna 27 may be removable from the antenna jack 26 so that the antenna 27 may be omitted in situations in which it is not needed. The antenna 27 may comprise various types of antennas, including but not limited to cellular phone antennas and RF radio antennas such as but not limited to RF24, RFMU, and the like.

As shown in FIG. 7, the computing unit 21 may include a power button 29. The power button 29 may be placed at various locations on the computing unit 21, such as on the lower end 23 of the computing unit 21 as shown in the figures. The operator 12 may press and hold the power button 29 for an extended period of time to power on and off the computing unit 21.

As shown in FIG. 7, the computing unit 21 may comprise a number of ports 110, 111, 112, 113, 114 on its lower end 23. The ports 110, 111, 112, 113, 114 are positioned on the lower end 23 of the computing unit 21 so that any cables or cords extend downwardly to minimize interference with maneuverability or vision of the operator when the computing unit 21 is in use.

The computing unit 21 may include a signal port 110 as shown in FIGS. 7 and 13. The signal port 110 is used to transmit an electrical signal to the personnel display unit 50, such as through use of a signal cable 62 connected between the signal port 110 of the computing unit 21 and a corresponding signal port 55 on the personnel display unit 50. The signal port 110 is used to both transmit and receive data and information from the personnel display unit 50. For example, graphical functions may be performed by the computing unit 21, with the display signal fed through the signal cable 62 to the personnel display unit 50. The computing unit 21 will also provide various other data and information to be displayed on the personnel display unit 50, such as operator 12 status, team status, locations, and the like.

The computing unit 21 may also include a power port 111 as shown in FIG. 7. The power port 111 is used to transmit electrical power to the personnel display unit 50, such as through use of a power cable 64 connected between the power port 111 of the computing unit 21 and a corresponding power port 56 on the personnel display unit 50. In this manner, the personnel display unit 50 need not have its own active power source, but instead may rely on power received from the computing unit 21. It should be appreciated, however, that in some embodiments, the personnel display unit 50 may be self-powered or may include its own auxiliary power supply to supplement power from the computing unit 21.

The computing unit 21 may also include a memory port 112 as shown in FIG. 7. The memory port 112 is used to receive an SD card 102 or other type of media. Such media may be used to update the firmware or software of the computing unit 21 or to upload data and/or information regarding any operators 12 being used in the field. For example, if a team is comprised of five operators 12 in the field, the SD card 102 or other media may preload the computing unit 21 with requisite data and information about those specific five operators 12. In some embodiments, the SD card 102 or other media may be utilized to store data collected by the computing unit 12.

The SD card 102 may be utilized to pre-program the computing unit 21. For example, each computing unit 21 may be pre-loaded with data and information related to the specific operator 12 operating that specific computing unit 21. The SD card 102 may be preloaded with the necessary information and data for that specific operator 12 and then loaded into the computing unit 21 via the memory port 113.

By way of example, the SD card 102 could be loaded with text documents with the parameters programmed into each file. For example, a first text file may include the personal information of the operator 12, such as name, age, sex, and the like. A second text file may store information on the configuration of the health protocols along with other software configurations. The third text file may store presets of network setup within the computing unit 21.

The computing unit 21 may also include a sensor port 113 as shown in FIG. 7. The sensor port 113 is connected to one or more physiological sensors 67 which are in contact with the operator 12 to provide real-time, continuous updates of the health information and status of the operator 12. As shown in FIGS. 3 and 4, a sensor cable 66 may be connected to the sensor port 113 of the computing unit 21, with the distal end of the sensor cable 66 having the sensor 67 which is put in contact with the operator 12 for physiological sensing.

The computing unit 21 may also include a universal serial bus (USB) port 114 such as shown in FIG. 6. The USB port 114 may be used to connect various accessories or subcomponents to the computing unit 21. The USB port 114 may also be used to connect various storage media, such as a USB hard drive, to the computing unit 21 for storage of data, updating firmware/software, or similar functions. The USB port 114 may also provide power to the computing unit 21, such as through a battery connected to the USB port 114.

It should be appreciated that the manner in which the computing unit 21 is powered may vary widely in different embodiments and should not be construed as limited. Any method known in the art for powering a processor 100, 101, 109 and/or subcomponents may be utilized. The power source may be internal to the computing unit 21 (such as by use of an internal power supply) or may be external, such as via the USB port 114.

In some embodiments, the computing unit 21 may rely upon both a primary power source and a secondary power source. The secondary power source may comprise a backup or reserve power source which only activates upon depletion of the primary power source. In this manner, each computing unit 21 may have reserve power to be relied upon if needed in dire situations.

One exemplary embodiment of primary and secondary power supplies may comprise the use of disposable batteries such as 9V or AA batteries as the primary power source. These types of disposable batteries are easy to carry in packs on the field to allow the batteries to be switched out as needed. An internal rechargeable battery such as a lithium rechargeable battery may be stored within the computing unit 21 to serve as the reserve, backup power supply for situations in which the operator 12 runs out of disposable batteries.

FIGS. 5-7 and 9 illustrate an exemplary embodiment of the computing unit 21 for use with the personnel system 20. As shown, this exemplary embodiment of the computing unit 21 comprises a pair of processors: a first processor 100 and a second processor 101. It should be appreciated that more or less processors 100, 101 may be utilized, and thus the exemplary embodiment shown in FIG. 5 should not be construed as limiting. When multiple processors 100, 101 are utilized, the processors 100, 101 may have redundancies so that one of the processors 100 can continue to function if the other processor 101 fails.

In multi-processor embodiments, each of the processors 100, 101 of the computing unit 21 may perform different functions. While exemplary division of functionality between the processors 100, 101 is discussed below, it should be appreciated that such discussions are merely for exemplary purposes only and thus should not be construed as limiting with respect to which functions are performed by which processor 100, 101. In other embodiments, a single processor, such as an ARM processor 109, may perform all of the functions of the computing unit 21.

In an exemplary multi-processor embodiment, the first processor 100 may be adapted to interface with the physiological sensor 67, the rip cord 40, and a radio transceiver 105. Various types of radio transceivers 105 may be utilized, including but not limited to an NRF2401 single-chip radio transceiver. The first processor 100 may be adapted to calculate the location and health status of the operator 12. The first processor 100 may also provide information to the personnel display unit 50 if worn through J1 Port 108.

In the same exemplary embodiment, the second processor 101 may be adapted to perform cellular phone and network functionality, including long-range communication functionality such as the RF24 Mesh and SIM900 cell phone communications protocols. The second processor 101 may also include a Bluetooth module 104 such as an HC-05 Bluetooth module.

The computing unit 21 may utilize an AT-Mega 2560 processor in some embodiments as the first processor 100 and/or the second processor 101. An AT-Mega 2560 may be utilized for both the first and second processors 100, 101 to perform processing functions. In other embodiments, different types of processors 100, 101, 109 may be utilized, such as a single ARM processor 109 as shown in FIGS. 14 and 15.

The first processor 100 may calculate the location and health status of any operators 12 in the field. The first processor 100 may also hold the software that intakes personnel information, location through GPS, and health information such as pulse. The second processor 101 may perform these same functions in some embodiments for redundancy. Continuing to reference FIG. 5, the computing unit 21 may include a GPS module 107 which is connected to either or both of the processors 100, 101. The GPS module 107 may be utilized to record the operator's 12 current location and transmit this information via serial communications to the processor 100, 101. In a preferred embodiment, a baud rate of 9600 bits per second may be utilized, though it should be appreciated that other baud rates may be utilized depending on the needs of the specific computing unit 21. By way of example and without limitation, an exemplary type of GPS module 107 for use with the computing unit 21 may comprise the Neo-6M GPS Module.

As shown in FIG. 5, the computing unit 21 may also comprise a sensor module 106. The sensor module 106 may be connected via serial connection to either or both of the processors 100, 101. The sensor module 106 is utilized to interface with both digital and analog pins to detect spikes in activity from the operator 12. The sensor module 106 may be adapted to detect heart rate of the operator 12. Extended times of limited or no activity which are detected by the sensor module 106 will signify to the software of the computing unit 21 that the operator 12 is possibly killed in action or otherwise disabled without the requirement of any input from the operator 12. By way of example and without limitation, an exemplary type of sensor module 106 for use with the computing unit 21 may comprise the AD8323 EKG module.

Continuing to reference FIG. 5, the computing unit 21 may also comprise a network communication device 103 adapted to utilize one or more cellular phone networks to transfer information from the computing unit 21 to the central network computer 134 and cloud database 120. The network communication device 103 may comprise a 32-bit microcontroller produced for the use of cellular reporting. The network communication device 103 may be utilized when operating in areas with cellular phone coverage provided by one or more cell phone towers 124. In areas without cellular phone coverage, other methods of communications may be utilized, such as use of the RF24 mesh network or use of radio communications. By way of example and without limitation, the network communication device 103 for use with the computing unit 21 may comprise a SIM900 Cellular Phone Network Communication Device.

The computing unit 21 may also comprise a radio transceiver 105 which allows for communications where cellular phone coverage may be limited or non-existent. The radio transceiver 105 may also provide communication redundancy in the event of failure of a cell phone tower 124 or other cellular communications hardware. The radio transceiver 105 may be adapted to communicate with other RF24 radio receivers for ranges up to 1,500 meters. The radio transceiver 105 allows the operators 12 with computing units 21 to communicate directly with each other and to relay information back to the cloud database 120. By way of example and without limitation, the radio transceiver 105 may comprise an NRF24 RF Transceiver.

The computing unit 21 may also comprise a Bluetooth module 104 for interfacing with various components, such as the laser targeting system 70, without the needs for cables or wires. The Bluetooth module 104 may be connected to a serial port on either or both of the processors 100, 101. Any components meant to interface with the computing unit 21 wirelessly, such as but not limited to the personnel display unit 50 and/or laser targeting system 70, may similarly include a Bluetooth module 77 so as to communicate with the Bluetooth module 104 of the computing unit 21.

Health status of the operator 12 may be determined by a combination of the rip cord 40 and the physiological sensor 67 in connection with the sensor module 106 of the computing unit 21. In some embodiments, either the rip cord 40 or the physiological sensor 67 may be utilized alone. Initial health settings may be loaded into the computing unit 21 via the SD card 102 in the memory port 112. The system will continuously monitor both the rip cord 40 circuit and the physiological sensor 67 to detect any variations from baseline.

### C. Weapons Integrated Targeting System

The system 10 may also include a laser targeting system 70 such as shown in FIGS. 17-19. The purpose of the laser targeting system 70 is to allow the operator 12 to report targets of interest by pointing their weapon 16 and pressing the lasing button. The laser targeting system 70 may mounted to the various locations on a weapon 16, such as to the bottom of the hand guard of any service rifle fitted with a modular rail system.

As shown in FIG. 19, the laser targeting system 70 may include a 16bit or 32bit processor 74, electronic compass 75, and laser range finder 73 module for integrating with the computing unit 21. A Bluetooth 77 module may be utilized for wireless communication with the computing unit 21. The laser targeting system 70 works by collecting the current azimuth of the weapon 16 from magnetic North, and determining the distance. Information is then sent to the computing unit 21 to be calculated with the current location from the GPS module 107 located inside the computing unit 21.

The laser targeting system 70 may include a laser emitter 72 for emitting a laser from the weapon 16 toward a target-of-interest. Although not shown, the laser targeting system 70 may include a button, switch, or other mechanism for activating or deactivating the laser emitter 72. The activation mechanism may be on the laser targeting system 70 or may be positioned remotely, such as on the personnel display unit 50, on the computing unit 21, or at various other locations which may be easily accessed by the operator 12.

The laser targeting system 70 may also include a laser range finder 73 which is fitted to measure the time of return from the projected laser to determine the distance of the target-of-interest. The laser range finder 73 may be fitted at various locations on the laser targeting system 70, such as at the front of the laser targeting system 70 in front of the electronic compass 75. The laser range finder 73 may comprise a laser receiver.

The electronic compass 75 will generally be positioned behind the laser range finder 73 so as to read measurements of the current magnetic field to determine the azimuth compared with true North. Software then performs calculations on the measurements to determine the true azimuth.

One or more processors 74 and associated software perform all processes within the laser targeting system 70 to include calculating the distance and azimuth from the sensor packages. After sensor information has been uploaded and calculated, the information is prepared in a command line and transmitted through the Bluetooth module 77 to the computing unit 21. Bluetooth is used to interface the laser targeting system 70 with the computing unit 21 to prevent the operator 12 from having wires protruding from their weapon 16. The laser targeting system 70 thus sends the processed data through Bluetooth to the host computing unit 21 for the completion of targeting data.

The laser targeting system 70 may be fitted to a weapon 16 such as a rifle that has a modular rail system fitted. The laser targeting system 70 is equipped with a laser range finder 73, electronic compass 75, and a stand-alone processor 74. A Bluetooth 77 module allows for the wireless integration with the computing unit 21.

The purpose of the laser targeting system 70 is to allow military, law-enforcement, and other security personnel to pin-point targets-of-interest with no extra gear. By mounting the laser targeting system 70 to their weapon 16, the laser targeting system 70 will reduce overall weight that the operator 12 has to carry. The laser targeting system 70 may be powered as a standalone system with its own power pack in some embodiments. In other embodiments, the laser targeting system 70 may draw power from the computing unit 21.

In one embodiment of the laser targeting system 70, data may be transmitted through a serial or wired mode of communication. The serial will communicate its information over hardwired communication. This system will be powered through the cable as well as transmitting targeting information through the hard line. This system will be distributed to system operators 12 who prefer hardwire connections over Bluetooth.

### D. Personnel Display Unit

As shown in FIGS. 11-16 and 25, the personnel monitoring and reporting system 10 may include a personnel display unit 50 which is worn by each operator 12 in the field. The personnel display unit 50 is used to display team and target data to the operator 12 wearing the personnel display unit 50. The personnel display unit 50 may be worn on the wrist of the non-weapon wielding arm of the operator 12, which allows for the display screen 52 to be viewed while the operator 12 is aiming his or her weapon 16 with the other arm.

The personnel display unit 50 is adapted to display information to the operator 12, including but not limited to team member location, health status, team targeting information, and objectives directed from dispatching or command software 122 discussed below. The location of team members or other friendly forces will be displayed relative to the operator's own location on the display screen 52 of the personnel display unit 50 at all times. This can provide operators 12 with a greater sense of situational awareness in fast-paced battle situations and operators.

As shown in FIG. 14, the personnel display unit 50 may be integrated with the computing unit 21, such as through use of signal and power cables 62, 64 connected between the computing unit 21 and the personnel display unit 50. The personnel display unit 50 may be powered by the processor(s) 100, 101, 109 of the computing unit 21 to generate graphics and power the screen to render all graphics. Information that may be displayed on the display screen 52 includes, but is not limited to, the operator's 12 location, team member's location and health status, targeting information, setup options, and personnel information.

FIG. 11 illustrates an exemplary embodiment of a personnel display unit 50 being worn on the wrist of a non-weapon wielding arm of an operator 12. As can be seen, the personnel display unit 50 comprises a display screen 52 which is visible to the operator 12. The display screen 52 may comprise a touch-screen in some embodiments. The display screen 52 may be color or black-and-white, and may be configured to operate in low-light conditions while limiting visible illumination.

As shown in FIGS. 11 and 12, the personnel display unit 50 may comprise one or more straps 53 for securing the personnel display unit 50 to the non-weapon wielding wrist of the operator 12. The shape, size, and number of straps 53 may vary in different embodiments and thus should not be construed as limiting in scope. Further, it should be appreciated that straps 53 may be omitted in some embodiments. In such embodiments, the personnel display unit 50 may be secured to the operator by other methods, such as by incorporation into clothing, use of adhesives, use of magnets, clasps, buttons, and the like.

FIG. 13 illustrates the side of the personnel display unit 50 which includes ports 55, 56 for interconnecting with the computing unit 21 to receive power and data such as graphical and location data. As shown, the personnel display unit 50 may include a signal port 55 which is connected by a signal cable 62 and signal connector 63 to a corresponding signal port 110 on the computing unit 21. Similarly, the personnel display unit 50 may include a power port 56 which is connected by a power cable 64 and power connector 65 to a corresponding power port 111 on the computing unit 21.

FIG. 14 illustrates the interconnection between an exemplary ARM processor 109 of a computing unit 21 and the personnel display unit 50. It should be appreciated that this is merely an exemplary embodiment, as the computing unit 21 may comprise additional processors 100, 101 in multi-processor embodiments. In such embodiments, both processors 100, 101, or one of the processors 100, 101, may be connected to the personnel display unit 50.

Continuing to reference FIG. 14, it can be seen that the ARM processor 109 of the computing unit 21 is connected serially to transmit a signal to the personnel display unit 50. A signal cable 62 is connected between the signal port 110 of the computing unit 21 and the signal port 55 of the personnel display unit 50. In this manner, data such as graphical data and team member locations may be continuously transferred in real-time to the personnel display unit 50 from the computing unit 21. Such a configuration negates the need for the personnel display unit 50 to have its own processor, though such a processor could be utilized in some embodiments.

Similarly, it can be seen that the ARM processor 109 of the computing unit 21 is connected serially to power the personnel display unit 50. A power cable 64 is connected between the power port 111 of the computing unit 21 and the power port 56 of the personnel display unit 50. In this manner, power from the computing unit 21 may be utilized to provide power to the personnel display unit 50. Such a configuration negates the need for the personnel display unit 50 to be self-powered, though in some embodiments the personnel display unit 50 may have its own primary or secondary power source.

FIG. 15 illustrates an exemplary interconnection between an ARM processor 109 of a computing unit 21 and a personnel display unit 50 via a J1 port 108 on the computing unit 21. The J1 port 108 allows the personnel display unit 50 to be directly connected to the computing unit 21. Generally, the computing unit 21 will have a signal port 110 and the personnel display unit 50 will have a signal port 55, with the signal ports 55, 110 being interconnected by a signal cable 62.

In the figures, the signal ports 55, 110 are illustrated as comprising female-connectors, with the male end being on the signal connector 63 of the signal cable 62. It should be appreciated that, in some embodiments, the reverse configuration could be utilized. It should be appreciated that a wide range of signal connectors 63 may be utilized comprising a range of pin numbers. In the exemplary embodiment shown in the figures, which is not meant to be limiting in scope, an exemplary signal connector 63 may be configured for an MDX 25 pin port.

With use of the J1 port 108, all graphical processing for the personnel display unit 50 may be performed by the one or more processors 100, 101, 109 of the computing unit 21. The J1 port 108 may be fitted to the one or more processors 100, 101, 109 such that a graphics engine may feed requests directly into the J1 port 108. In this manner, the personnel display unit 50 may rely solely on the one or more processors 100, 101, 109 for all graphical processing. It should be appreciated, however, that in some embodiments the personnel display unit 50 may include its own processor(s) for graphics processing and thus not be reliant upon the computing unit 21 for such functionality.

FIGS. 3, 4, and 10 illustrate the cabling and wiring necessary for a wired connection between the computing unit 21 and the personnel display unit 50. It should be appreciated that, in some embodiments, the computing unit 21 and personnel display unit 50 may be wirelessly connected. For example, in some embodiments, the personnel display unit 50 may have its own processor and Bluetooth module for communicating with the computing unit 21.

In the exemplary wired embodiment shown in FIGS. 3, 4, and 10, it can be seen that the various cables 62, 64, 66 have been seated in a conduit jacket 60. This prevents loose cables 62, 64, 66 from interfering with the operator 12 and reduces clutter. In such an embodiment, the signal cable 62 and power cable 64 connected between the computing unit 21 and personnel display unit 50 may be seated in the conduit jacket 60. Similarly, the sensor cable 66 which connects to the sensor port 113 of the computing device 21 may be seated in the conduit jacket 60.

The signal cable 62 is connected at its first end to the signal port 110 of the computing unit 21. The signal cable 62 then extends through the conduit jacket 60. The signal cable 62 exits the conduit jacket 60 to be connected to the signal port 55 of the personnel display unit 50 by a signal connector 63 such as shown in FIGS. 10 and 14.

Similarly, the power cable 64 is connected at its first end to the power port 111 of the computing unit 21. The power cable 64 then extends through the conduit jacket 60. The power cable 64 exits the conduit jacket 60 to be connected to the power port 56 of the personnel display unit 50 by a power connector 65 such as shown in FIGS. 10 and 14.

The sensor cable 66 is not connected to the personnel display unit 50, but instead terminates into a physiological sensor 67 that is generally in physical contact with the body of the operator 12. In an exemplary embodiment, the sensor cable 66 may be connected at its first end to the sensor port 113 of the computing unit 21. The sensor cable 66 may extend through the conduit jacket 60 with the signal and power cables 62, 64 or may be routed separately. In either case, the sensor cable 66 terminates into a physiological sensor 67 that is contact with the skin of the operator 12. By way of example, the physiological sensor 67 could be positioned to contact the armpit, chest, or wrist of the operator 12, among various other locations on the operator's 12 body.

FIG. 16 illustrates an exemplary display screen 52 of a personnel display unit 50. FIG. 25 illustrates an alternate embodiment of a display screen 52 and personnel display unit 50 that includes controls 54 for manipulating the interface. The controls 54 may be comprised of hard buttons or may comprise graphical indicia on a touch screen.

The display screen 52 is visible to the operator 12 in the field to provide valuable information about the operator's 12 health and location as well as the health and location of other team members in the field. It should be appreciated that the exemplary interface shown on the exemplary display screens 52 of FIGS. 16 and 25 are merely for illustrative purposes and thus should not be construed as limiting in scope. The interfaces shown on the display screens 52 may vary widely between different embodiments due to different preferences of different operators 12 and the needs of different types of operations.

In the exemplary interface shown in FIGS. 16 and 25, it can be seen that the display screen 52 has been split into three discrete sections: a personal status display 57, a team status display 58, and an area display 59. The personal status display 57 includes information about the operator 12 and the computing unit 21, such as but not limited to the health status of the operator 12 (derived from the physiological sensor 67), the location of the operator 12 (derived from the GPS module 107 of the computing unit 21), and whether the SD card 102 has been properly loaded into the computing unit 21.

The team status display 58 includes information about the health status of any team members operating in the field with the operator 12. The manner in which the health status of fellow team members is conveyed to the personnel display unit 50 is discussed in more detail below. Indicators may be utilized to provide quick at-a-glance health status information about each team member for the operator 12 wearing the personnel display unit 50.

The area display 59 includes information about the status and location of any team members within range of the computing unit 21. The area display 59 may include geographical information, such as direction, terrain information, and points of interest. The area display 59 may also have an indicator for each team member working with the operator 12 in the field, as well as the current location of the operator 12. In the exemplary figure, team members may be represented as circles and health status is represented by rectangles and half-moon symbols. Different types of indicators or icons may be utilized to quickly convey information to the operator 12. For example, colors may be utilized to quickly and easily indicate health status of team members. A green icon may indicate a healthy team member, a yellow icon may indicate a team member in danger, and a red icon may indicate a disabled team member.

Icons may be included to denote areas of interest, topography features, and other information about the area in which the operator 12 is operating. Audible warnings may be provided by the personnel display unit 50 or computing unit 21 to urge the operator 12 to view the display screen 52 for important information. For example, if a team member is disabled, an audible warning may be emitted to encourage the operator 12 to view his/her display screen 52 to locate the team member in need of assistance. Further, the display screen 52 may identify targets which have been highlighted by a team member, such as through use of a laser targeting system 70. Finally, the display screen 52 may include distance information to other team members, areas of interests, or identified targets.

### E. Network Connectivity

In the field, there will typically be a team of operators 12 each having their own personnel system 20 including a computing unit 21 and optional personnel display unit 50 and laser targeting system 70. The personnel monitoring and reporting system 10 is configured such that the team of operators 12 may communicate location and health information not only to each other, but also to a central network computer 134 operating software such as dispatch software 122 or command software 136 and which may include databases such as a cloud database 120 or a relation database 135.

The computing units 21 may utilize a number of communications protocols to allow for network connectivity both with central command and with other computing units 21. Each computing unit 21 may include multi-factor communication standards or, in some embodiments, may be limited to only one communications protocol. The computing units 21 are configurable to work with multiple communications protocols or only one communications protocol.

By way of example, each computing unit 21 may be configured for cellular phone communications, radio frequency mesh networks, and long range radio communications. These communications protocols may be powered by the serial ports on the one or more processors 100, 101, 109 of the computing unit 21. These modes of communications may be switched on and off with respect to each computing unit 21 to meet the needs of different operations and operators 12. Thus, each computing unit 21 is fully customizable to communicate using one or all of the communications protocols and modes discussed herein.

FIGS. 20 and 22 illustrate a mesh networking communications protocol for use by a plurality of computing units 21 in the field. The mesh networking protocol utilizes mesh networking without a central transmission or receiving point, similar to peer-to-peer distribution of files through torrents. Mesh networking provides an effective method of communications between operators 12 in areas where no Wi-Fi or cellular phone coverage can be used. When using the mesh networking protocols, each computing unit 21 stores information of other friendly and enemy assets in the battle space.

FIG. 20 illustrates an exemplary diagram of a mesh networking communications protocol in use. As shown in FIG. 18, each circle represents a radio node for each of the operators 12 in the field. Each square represents the collection of information within the computing unit 21 of each operator 12 and the text above the operator 12 represent the current information of that operator 12. Lines represent the communications between the computing units 21 in the field.

The radio nodes create an expansive network which allows for more coverage as the operators 12 span out, even without a central transmission point. Each radio node may have a range of, for example, 1,500 meters which allows each computing unit 21 to communicate with other computing units 21 in range nearby. This type of distributed networking eliminates the risk of a single point of failure affecting communications between operators 12 in the field.

FIG. 21 illustrates an alternate communications protocol which utilizes a squad reporting radio (SRR) 130. Each unit or team of operators 12 is assigned to an SRR 130. Thus, each computing unit 21 assigned to a specific SRR 130 will communicate various data and information to the assigned SRR 130. The SRR 130 will report status such as health and location data from any computing units 21 in range to an SRR network 132.

The SRR 130 may be small in size using long range radio systems. The SRR 130 may utilize a whip antenna to relay signals up to five miles. In other embodiments, the SRR 130 may be mounted to a vehicle with a range of up to 8 miles of line of site communications. The SRR 130 may be installed on one or more computing units 121 using an antenna jack 26. The use of SRR's 130 creates a large scale SRR network 132 which is designed to transmit data over extreme distances. The SRR's 130 may also be fitted with a lower mesh network so that computing units 21 may communicate to the radio system.

After the data has reached the root node of the SRR network 132, the information is then related to a network computer 134 with access to the server in which the relation database 135 resides. This can be accomplished by either SQL or NoSQL servers. Command software 136 then pulls information from the relation database 135 to populate mapping information in the command software 136. The command software 136 displays the physical location, health status, and unit of the operators 12 in the field. The command software 136 can also perform statistics on this data to aid commanders in making decisions as events unfold.

FIG. 22 illustrates operation of the mesh network protocol using an SRR 130. As can be seen, any computing units 21 within the range of the mesh network report directly to the SRR 130. Computing units 21 outside of the range of the mesh network report to the closest of the other computing units 21 which are inside the range of the mesh network.

FIG. 23 is illustrates a sub-operation of the mesh communications network. A mirror resides in the SRR network's 132 software and serves as a mini data collection for recently reported information. Every time a computing unit's 21 message is reported to the SRR network 132, the information is logged within the mirror in the form of a database table. This collection of information is then echoed or sent back to the other computing units 21 in the mesh network so that personnel display units 50 can be updated information of fellow team members. Additionally, the mirror decides which information is to be transmitted at what time to the SRR network 132. By controlling priority of data transfer, long range networks can be kept from being slowed down from over reporting. The mirror only reports the information when the data has changed.

FIG. 24 illustrates yet another communications protocol for use with the personnel monitoring and reporting system 10. This particular communications protocol is best-suited for urban areas in which cell phone towers 124 are in range of the computing units 21, but suffers from a single point of failure.

As shown in FIG. 24, each computing unit 21 communicates, such as through a network communication device 103 such as an antenna, with a cell phone tower 124. The cell phone tower 124 transmits data to the cloud database 120 to update the status and information on each of the computing units 21.

The same data from the cloud database 120 is continuously fed back to the computing units 21 by the cell phone tower 124. Dispatch software 122 or command software 136 as discussed below may also be in communication with the cloud database 120 and cell phone tower 124 so that operations may be managed from a central location.

### F. Command Software

The command software 136 is a front-end application used by dispatchers and high level leadership. The command software 136 displays location and health data in a detailed map of the city, area, or battle space while performing statistics and calculations on current events. The command software 136 can also integrate with other first responder units such as medical personnel and ambulances for the purpose of ease of sharing information.

The command software 136 also allows for the pushing of digital information and data to the computing units 21 through either cellular networks or other radio networks through application servers. Such a feature allows for the pushing of directives to police officers and soldiers, such as when responding to a downed operator 12.

The command software 136 may have connections to the database 120, 135 which holds current information on computing unit 21 uplinks. This information may be used to display detailed personnel information on a map while performing calculations and processing. The command software 136 is a fully equipped user interface which integrates with application and database servers 120, 135 to represent current field information. The command software 136 is intended for use by dispatchers, mission planners, and upper level leadership of the organization.

The command software 136 will display all individuals wearing computing units 21 information that they spot to include target of interests. The command software 136 is also designed to integrate with ambulance and first respondesr by pushing internet resources to their network in sharing information reported by the operators 12 wearing computing units 21.

The command software 136 uses maps, graphical rendering of objects which represent operators 12, and any associated asset such as another first responder unit. The command software 136 may also have built in artificial intelligence so as to make recommendations to the operator of the command software 136 in making life and death decisions. The command software's 136 primary objective is reducing the loss of life in the line of duty.

The cloud database 120 may be utilized to store, process, receive, and/or transmit data and information between the various operators 12 in the field using computing units 21 and the command structure, whether the command structure be centrally located in one location or distributed over a geographical area. An application server 134 may be utilized to operate the command software 136 which relies upon the data and information stored and continuously updated on the cloud database 120. The cloud database 120 may be stored on the same application server 134 or may be stored on one or more external computer systems.

Generally speaking, the application server 134 will receive, store, process, and update coordinates received from any number of computing units 21 in the field. The application server 134 may receive the coordinates in the form of data transferred via any of the communications protocols previously discussed. For example, the application server 134 may maintain a continuous communicative interconnection with the SRR 130, with the computing units 21 individually reporting to the SRR 130.

The application server 134 will maintain current coordinates for each of the operators 12 in the field using computing units 21 and continuously transmit updated coordinates to all operators 12 either directly or through the SRR 130. The application server 134 will continuously update the cloud database 120 with updated coordinates as they are received.

The computing units 21 will generally process the coordinates received from the application server 134, such as through a cellular tower 124 or SRR 130, to update the display screen 54 of the personnel display unit 50 with status, location, and other information of the other friendly operators 12 in the field. Thus, the processor(s) 100, 101, 109 of the computing units 21 will perform most processing functions, including graphical processing for the personnel display unit 50.

### G. Operation of Preferred Embodiment

The systems and methods described herein may be utilized to give senior leadership of commanding forces or dispatched units a greater sense of situational awareness through field data collection. Each service member (operator 12) is equipped with a computing unit 21 which may be attached to his/her body armor 13. The computing units 21 each collect information such as personnel information, location, and health status.

In this manner, senior leadership down to field leadership are given the tools necessary to view current unfolding events and display them in a manner that is easy to understand. Leadership may utilize specialized command software 136 tuned to operate with the computing units 21 in the field to display real-time information to leadership at a central location. Each operator 12 in the field may also equip a personnel display unit 50 on their non-weapon wielding arm to quickly view location and status information in the field.

The systems and methods described herein further keep accountability of all supporting assets to include medical, air support, and combat vehicles. This assists commanders with sending support to operators 12 in need. Back-end software will determine the best tool for the job based on the response time and purpose of the supporting assets, with the goal of reducing response times of both medical and support assets and using them to their fullest advantage.

The computing unit 21 provides a number of features, including determining health status by two-factor (sensor 36 and rip cord 40) systems, identifying location through GPS or other location services, multiple factors of communication, redundant system design, and the ability to communicatively interconnect, through wired or wireless connections, with modifications to enhance the capabilities of the computing unit 21, such as but not limited to a personnel display unit 50 and a laser targeting system 70.

The command software 136 provides a number of features to command, including identification of the locations and health statuses of all operators 12 in the field of operation, storing information relating to aircraft and ground assets, and providing recommendations through artificial intelligence to assist commanders in making decisions to decrease loss of life of friendly forces.

In use, each operator 12 may be fitted with a computing unit 21. The computing unit 21 may be worn on the body of the operator 12, such as by being supported by the mesh fabric common on body armor 13. Each computing unit 21 collects information from the operator 12 based on sensor 67 readings, rip cord 40 status, the GPS module 107, and user input. The computing units 21 may communicate with each other and with central command using the protocols discussed above.

Determining health status of each operator 21 works by factoring two elements. The heart beat sensor 67 determines if the individual is a possible KIA, and the assistance rip cord 40 indicates times of danger or when assistance is urgently needed. If the operator 12 becomes injured, they are able to pull the rip cord 40 which configures the computing unit 21 to report the operator 21 is injured. If the heart beat sensor 67 has not in taken a pulse in a considerable amount of time, the personnel will be reported as a possible KIA.

The laser targeting system 70 integrates with the computing unit 21 to share azimuth and range information to the processor(s) 100, 101, 109 of the computing unit 21. The computing unit 21 will then request user input from the operators 12 about the targeted object. The personnel display unit 50 may then specify what is being spotted in the network. The laser targeting system 70 and personnel display unit 50 thus work in conjunction with each other. After the information is complete, the computing unit 21 stores the information, and transmits the targeted information through the network to be stored in a database 120, 135.

After processing and calculating the operator's 21 health status and location, the data is sent to the application server 134 using any of the communications protocols discussed previously. The application server 134 processes querying commands to alter the database 120, 135 which represents current events. The database 120, 135 holds all real-time information about its operators 12 and is stored within the cloud so that it may be accessed from anywhere.

When querying commands are received by the application server 134, software then alters the database 120, 135 to represent current field events. This allows for the holding of current information and can be updated frequently. This database 120, 135 is also a relational database meaning it takes less memory in storage to store all data.

On computers with the command software 136 installed have the clearance to access the application server 134. Dispatchers or commanders can view the information being sent from the computing units 21 in real-time. This works by pulling the real-time information from the database 120, 135 and representing each operator's 12 health status and GPS location on a detailed map of the city or battle space.

Back end software processes are also carried out through the user of multi-threading. In a separate thread of the command software 136, artificial intelligence is encoded into the command software 136 which is programmed to finding multiple avenues of approach to a situation to reduce response times. This can be selecting a police officer that is closer to a scene of a crime or an ambulance department that is better equipped to deal with the medical emergency.

The command software 136 can also push resources to other organization through the use of HTTP resources. This means emails of hospitals can setup hub centers or use email addresses to receive detailed information about the incoming injured personnel to best prepare. HTTP resources can push plain text or complex data through internet resources and embedded within the software of Guardian are code structures designed to handle and distribute these requests at the decision of the dispatcher.

The type of network communications protocol utilized by the computing units 21 in the field may be selected prior to or during operations. As previously mentioned, the computing units 21 may be configured to operate using a number of communications protocols, such as but not limited to cellular phone systems, radio frequency communications, mesh networks, and the like. In some embodiments, each computing unit 21 may be configured to operate using one communications protocol. In other embodiments, the computing units 21 may be configured to operate using multiple communications protocols.

In an exemplary embodiment, software may be utilized to control which of the communications protocols is active within the computing unit 21 at a given time. By way of example, text files in the SD card 102 may be used to configure various features of the computing unit 21, including the activation or deactivation of certain communications protocols. Further, the SD card 102 may be utilized to activate or deactivate various other features/modules of the computing unit 21, such as the sensor 67, radio transceiver 105, Bluetooth module 104, GPS module 107, and the like. For example, it may be desirable in firefighting operations to deactivate the EKG pads 67a, 67b, 67c. In such circumstances, a text file may be loaded into the SD card 102 which deactivates the EKG pads 67a, 67b, 67c.

The SD card 102 may also store various information, including meta data. The SD card 102 may have its own internal database which is updated periodically to allow for data redundancy. For example, if a computing unit 21 loses connectivity with the SRR 130 for a short period of time, data will continue to be logged and saved in the SD card 102. When connectivity is restored, the same data will be transferred to update the SRR 130. Thus, even when out of range of the SRR, data about the operator 12 may be stored in his/her own personal computing unit 21 until such time as connectivity is restored.

Unless otherwise defined, all technical and scientific terms used herein have the same meaning as commonly understood by one of ordinary skill in the art to which this invention belongs. Although methods and materials similar to or equivalent to those described herein can be used in the practice or testing of the personnel monitoring and reporting system, suitable methods and materials are described above. All publications, patent applications, patents, and other references mentioned herein are incorporated by reference in their entirety to the extent allowed by applicable law and regulations. The personnel monitoring and reporting system may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. A personnel monitoring and reporting system, comprising:
a computing unit adapted to be secured to an operator, the computing unit comprising a processor and a transceiver, wherein the computing unit comprises a pair of electrical contacts;
a physiological sensor communicatively interconnected with the computing unit, wherein the physiological sensor is adapted to be in physical contact with the operator, wherein the physiological sensor is adapted to transmit one or more health conditions of the operator to the computing unit; and
a rip cord removably connected to the computing unit, wherein the rip cord comprises a conductive strip adapted to electrically connect the pair of electrical contacts of the computing unit, wherein the rip cord is adapted to be removed from the computing unit such that the conductive strip no longer electrically connects the pair of electrical contacts of the computing unit, wherein the computing unit is adapted to transmit a distress signal through the transceiver when the pair of electrical contacts are not electrically connected.

2. The personnel monitoring and reporting system of claim 1, wherein the computing unit comprises a sensor port, wherein the physiological sensor is connected to the sensor port of the computing unit by a sensor cable.

3. The personnel monitoring and reporting system of claim 1, wherein the physiological sensor comprises a pulse detector.

4. The personnel monitoring and reporting system of claim 1, wherein the computing unit comprises a memory slot for receiving a memory card, the memory card being adapted to update software of the computing unit.

5. The personnel monitoring and reporting system of claim 1, wherein the transceiver is comprised of a Bluetooth transceiver.

6. The personnel monitoring and reporting system of claim 1, wherein the transceiver is comprised of a radio antenna.

7. The personnel monitoring and reporting system of claim 1, wherein the transceiver is comprised of a cellular communications unit.

8. The personnel monitoring and reporting system of claim 1, further comprising a personnel display unit communicatively interconnected with the computing unit, wherein the personnel display unit is adapted to be worn by the operator.

9. The personnel monitoring and reporting system of claim 8, wherein the personnel display unit comprises a display screen for displaying information about the operator.

10. The personnel monitoring and reporting system of claim 9, wherein the display screen is further adapted to display a status and location of one or more team member operators operating a secondary computing unit.

11. The personnel monitoring and reporting system of claim 8, wherein the personnel display unit is adapted to be worn on a wrist of the operator.

12. The personnel monitoring and reporting system of claim 8, wherein the computing unit comprises a first power port and the personnel display unit comprises a second power port interconnected by a power cable such that the personnel display unit is powered by the computing unit.

13. The personnel monitoring and reporting system of claim 12, wherein the computing unit comprises a first signal port and the personnel display unit comprises a second signal port interconnected by a power cable such that the computing unit controls a display screen of the personnel display unit.

14. A personnel monitoring and reporting system, comprising:
a computing unit adapted to be secured to an operator, the computing unit comprising a processor and a transceiver;
a physiological sensor communicatively interconnected with the computing unit, wherein the physiological sensor is adapted to be in physical contact with the operator, wherein the physiological sensor is adapted to transmit one or more health conditions of the operator to the computing unit;
a personnel display unit communicatively interconnected with the computing unit, the personnel display unit comprising a display screen for displaying a location and status of the operator;
a weapon adapted to be carried by the operator; and
a laser targeting system connected to the weapon, the laser targeting system comprising a laser emitter such that the operator may identify one or more targets with the laser targeting system.

15. The personnel monitoring and reporting system of claim 14, wherein the laser targeting system comprises a laser range finder for detecting a range to any of the one or more targets identified with the laser targeting system.

16. The personnel monitoring and reporting system of claim 15, wherein the laser targeting system comprises an electronic compass.

17. The personnel monitoring and reporting system of claim 14, wherein the laser targeting system is wirelessly connected to the computing unit.

18. A method of monitoring personnel during an operation, comprising the steps of:
equipping a plurality of operators with a plurality of computing units, wherein each of the operators wears one of the plurality of computing units, wherein each of the plurality of computing units comprises a transceiver having an effective range;
identifying any of the plurality of computing units in the effective range by a first computing unit of the plurality of computing units;
transmitting a location information of any of the plurality of computing units identified in the effective range by the transceiver of the first computing unit to a squad reporting radio; and
transmitting the location information of any of the plurality of computing units identified in the effective range by the squad reporting radio to a central control unit.

19. The method of claim 18, further comprising the step of transmitting the location information of any of the plurality of computing units not in the effective range of the first computing unit to the squad reporting radio by the central control unit.

20. The method of claim 19, further comprising the step of transmitting the location information of any of the plurality of computing units not in the effective range of the first computing unit to the first computing unit by the squad reporting radio.
